(19) United States
(12) Patent Application Publication
(10) Pub. No.: US 2023/0056266 A1
(43) Pub. Date: Feb. 23, 2023

(54) LIGHT TRANSMISSIVE COMPOSIT COMPONENT AND METHOD OF FORMING THE COMPONENT

(71) Applicant: Luke Latham Moorhouse HEATON, London (GB)

(72) Inventor: Luke Latham Moorhouse HEATON, London (GB)

(21) Appl. No.: 17/792,365

(22) PCT Filed: Jan. 15, 2021
(86) PCT No.: PCT/GB2021/050089

§ 371 Date: Jul. 12, 2022

(30) Foreign Application Priority Data
Jan. 17, 2020 (GB) 2000724.1

(51) Int. Cl. F21S 11/00; C09J 199/00; C12N 1/14; E04C 1/42; E04C 2/16; G02B 6/04; B28B 23/00

(52) U.S. Cl. CPC F21S 11/007; C09J 199/00; C12N 1/14; E04C 1/42; E04C 2/16; G02B 6/04; B28B 23/0037

(57) ABSTRACT

A method of forming a light transitive composite component. The method comprises inserting at least one light transmitting component 3, an organic substrate 6 and bonding agent 10 into a mould 1. Allowing the bonding agent 10 to mature to bond both the light transmitting component 3 and the organic substrate 6. Treating the contents of the mould with either heat or chemicals to terminate the maturation process of the bonding agent. The organic substrate is preferably a woody substrate while the bonding agent is preferably fungal mycelium.

The present invention relates to a light transmissive composite component and to a method of forming the component.

The present invention is directed to a light transmissive composite component suitable for use as a non-load bearing structural or semi-structural component such as an inf ill wall panel or roof tile. However, the component may also be used in other applications such as movable partitions, light fittings, light shades or luminaires.

Light transmissive composite components are known in the art which are made of concrete matrix embedded with glass optical fibres. Such components are useful as they act as structural or semi-structural components while still allowing the transmission of light. The transmission of natural light folding through a structural or semi-structural component can reduce the energy consumption of a building as it can reduce the amount of artificial light required as well as being more thermally efficient than providing additional windows. Such components also allow light to be transmitted into a building through walls that cannot contain windows, due to privacy rights of the overlooked space.

Such light transmissive composite components can also be made into aesthetically pleasing shapes that can also be used to produce unusual and attractive light effects.

The present invention aims to provide a light transmissive composite component which allows more flexible designs and is more environmental friendly.

According to a first aspect of the present invention, is provided a method of forming a light transmissive component according to claim 1.

Rather than the concrete of the prior art, the present invention uses a matrix which is formed of an organic substrate, a light transmitting component and a bonding agent that adheres to both the organic substrate and the light transmitting component. The organic substrate is intended to provide a structural or woody substrate to which the bonding agent bonds.

The bonding agent preferably contains fungal mycelium, but may also consist of an adhesive mucilage containing polysaccharides, oligosaccharides or other soluble polymers, such that the substrate and mucilage form a well lubricated, pourable mixture, but when the mucilage starts to dry, the polymers in the mucilage bond with each other and the surrounding materials through a series of dehydration reactions, forming an adhesive crust.

Fungal mycelium is composed on a network of micro-filaments known as hyphae which have a natural tenancy to grow into microscopic gaps or cracks. Hyphae digest and bond to the surfaces of the organic material, effectively acting as a natural, self-assembling glue. This growing of filaments allows the material to readily fill more complex shapes as well as providing improved bonds allowing more complex arrangements of light transmitting components.

The method provides environmental benefits in that it reduces the mass of material transported for construction as the organic substrate can readily be sourced close to the site where the method is carried out. Further, the organic substrate can be locally available organic waste material, that would otherwise incur costs associated with disposal. The fungal mycelium may be grown in situ. The material also has good fire retardant and thermal degradation properties making it ideally suitable as a structural or semi-structural product.

While using fungal mycelium as the principal bonding agent does take some time (typically a matter of weeks), this can be done in the absence of any light or other energy such that the components can be formed in large stacks in unlit facilities.

The organic substrate may be any material that the bonding agent can adhere to. In practice, there are a wide range of options allowing use of whatever is most convenient, or available locally. Typical substrate materials may include leaf litter, wood chips, twigs, weeds, grass cuttings, cardboard, agricultural waste, corn husks, rice husks and straw. Organic waste may be boiled and squeezed to reduce or remove soluable nutrients, with the nutrients returned to the soil while lignin, cellulose, hemicellulose and other structural or fibrous materials are dried and used as a substrate for the present invention.

The organic substrate should be dry enough to minimize bacterial decomposition. This can be achieved by placing a thin layer of weeds, grass cutting or other organic substrates with a high water content on a hot surface. The organic substrate should also be sterilized, killing any organisms that may digest or degrade either the substrate or the bonding agent.

This may be achieved by dipping the dry organic substrate into a hot tub of mucilage. Mucilage may contain nutrients such as sugars or amino acids, as enriching the substrate with such nutrients will accelerate fungal growth. It may also contain polysaccharides, oligosaccharides or other soluble polymers, such that the substrate and mucilage form a well lubricated, pourable mixture, but when the mucilage dries, it forms an adhesive crust. Mucilage may also contain compounds that enhance the fire retardant properties of the composite material.

The properties of the composite component can also be tuned with the presence of one or more inorganic compounds such as sand, clay, gravel, plastic waste or rubble. These can be added to increase the thermal mass of the product or to increase the thermal and acoustic insulation. Thermal mass adjacent to the light transmitting plastic is particularly desirable, as it will store and slowly release the heat generated by solar radiation, which enters the present invention through the light transmitting plastic. Substrates with a relatively large proportion of clay or other high thermal mass material may be sprayed onto the light transmitting plastic first, followed by substrate containing a smaller proportion of high thermal mass material. Addition of waste materials may also reduce the costs that would otherwise be incurred disposing of these materials.

The light transmitting component may, for example, be an optical fibre. Preferably, the optical fibre will be plastic as this is a flexible component which can take up a curved path within the mould to allow well controlled light paths to be defined through the component.

This works well with the ability of fungal mycelium to grow into small gaps allowing multiple complex light paths to be formed. The transmissive component can also be a plastic layer wherein a portion of one face of the component is moulded as a layer of light transmissive plastic. This provides a light transmissive surface at an outer face of the component.

Preferably, the light transmissive components are a combination of the plastic optical fibres and the plastic layer.

Light transmitting surfaces may be composed of multiple plastics, with a thin outer layer of plastic chosen to increase internal reflection, reduce moisture absorption, or to reflect ultra-violet light which would otherwise degrade the plastic. The surface of the component may also be coated with a resin in order to improve the waterproof properties of the component.

Fungal mycelium responds to mechanical damage with proliferative growth, such that, preferably, the substrate is mechanically compacted one or more times during the growth phase. This also allows the density of the finished components to be controlled or optimized.

Most fungi are capable of digesting their own biomass to fuel further growth. If unchecked, this capacity for autophagy may result in a net decrease in fungal biomass within the component. To prevent this undesirable outcome, fungi should be killed once they have grown to fill the mould. This may be achieved by heating the mould, and prolonged warming to a temperature as low as 40° C. may be sufficient to mature the bonding agent, by drying the mucilage and killing the fungal mycelium. In many applications, it may be more efficient to dry the mucilage and kill the fungal mycelium by briefly heating the mould to 60° C. Alternatively, maturation of the fungal mycelium may be arrested by the addition of small quantities of fungicidal compounds such as 2-aminoisobutyric acid.

The present invention also extends to a composite component according to claim 14. This component may be provided with the preferred features as set out above.

Examples of method and component in accordance with the present invention will now be described with reference to the accompanying drawings in which:

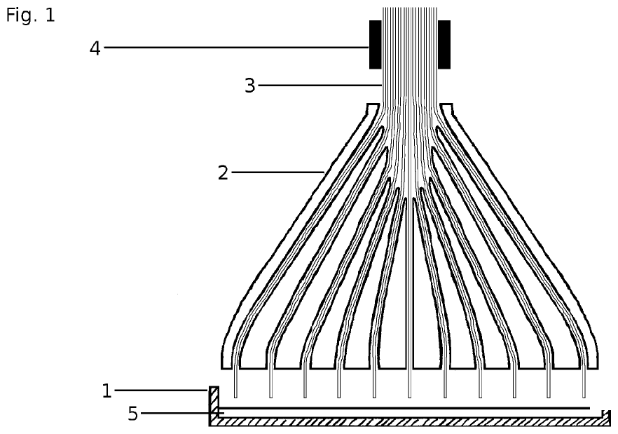

FIG. 1 is a schematic cross-section of a first stage in the process of forming a first component;

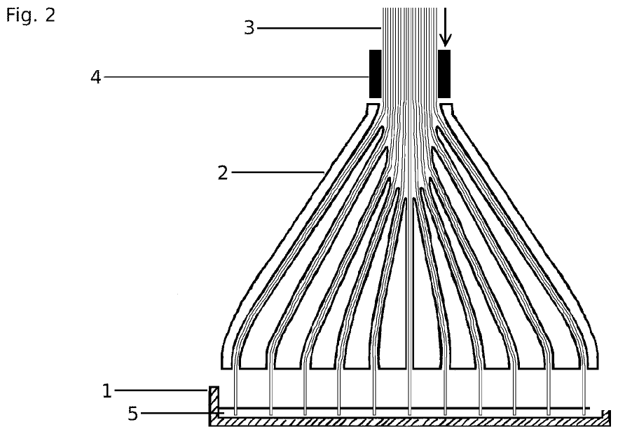

FIG. 2 is a view similar to FIG. 1 showing a second stage of the process;

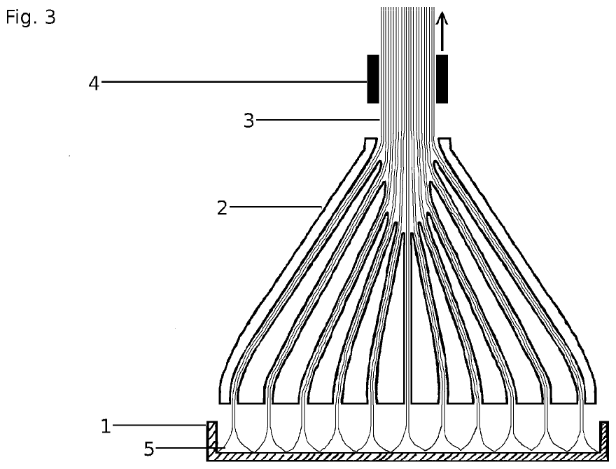

FIG. 3 is a view similar to FIG. 2 showing a third stage of the process;

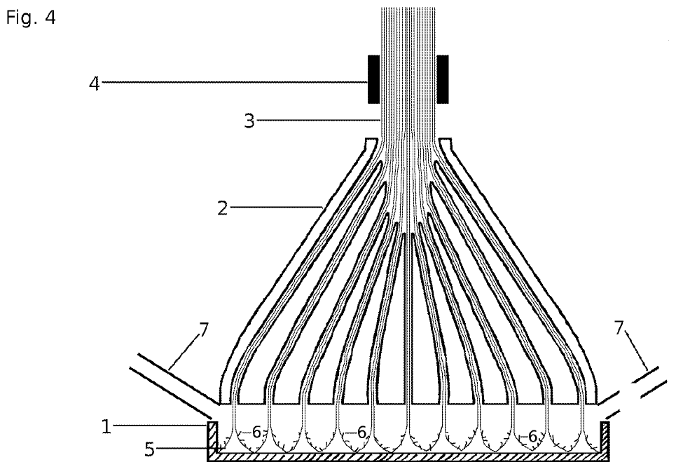

FIG. 4 is a view similar to FIG. 3 showing a fourth stage of the process;

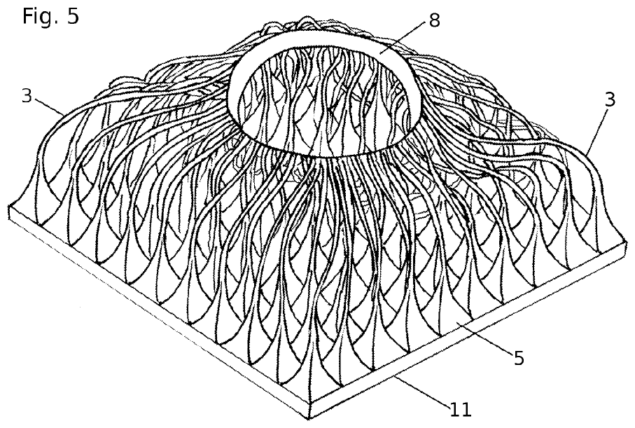

FIG. 5 is a isometric view showing the component after the completion of a fifth stage of the process;

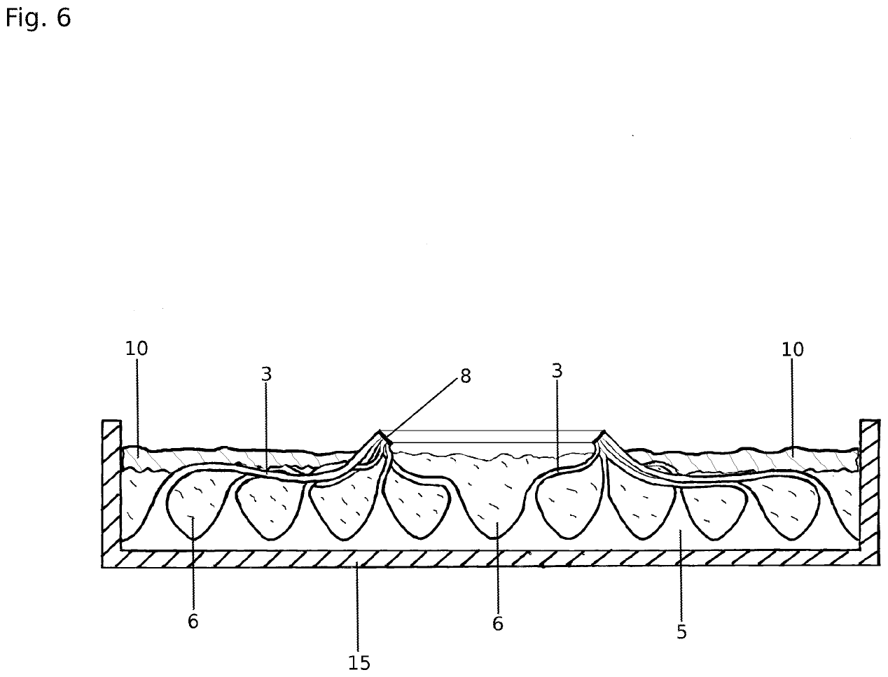

FIG. 6 is a view similar to FIG. 4 showing a sixth stage to the method;

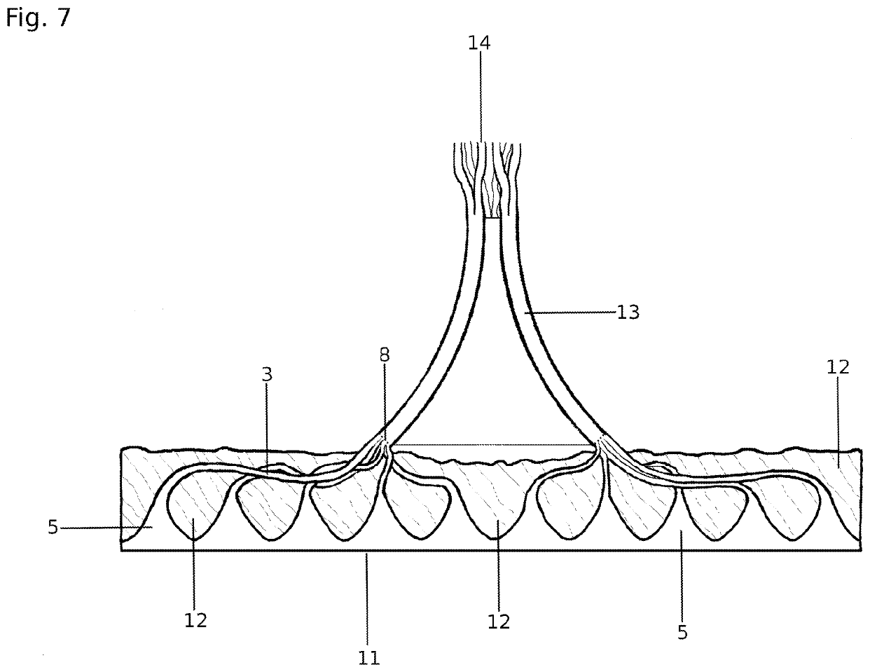

FIG. 7 is a view similar to FIG. 6 of the finished component. FIG. 7 also shows an optical junction that attaches to the finished component, which directs the light emanating from the finished component into fibre optic cables.

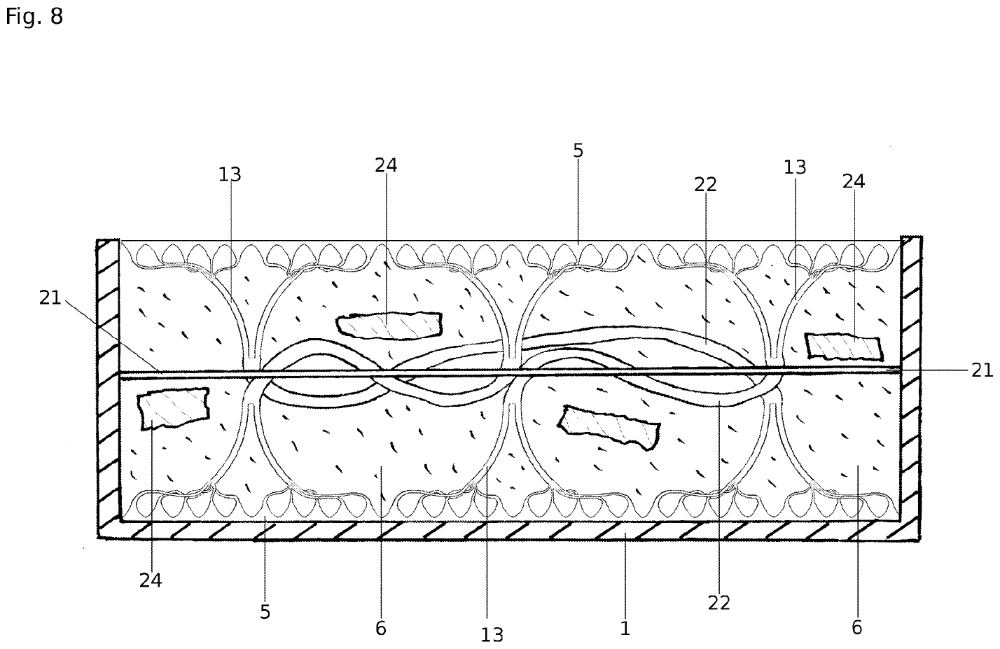

FIG. 8 is a cross-sectional view of a second component made of a plurality of sub-components at the same stage of processing in FIG. 6;

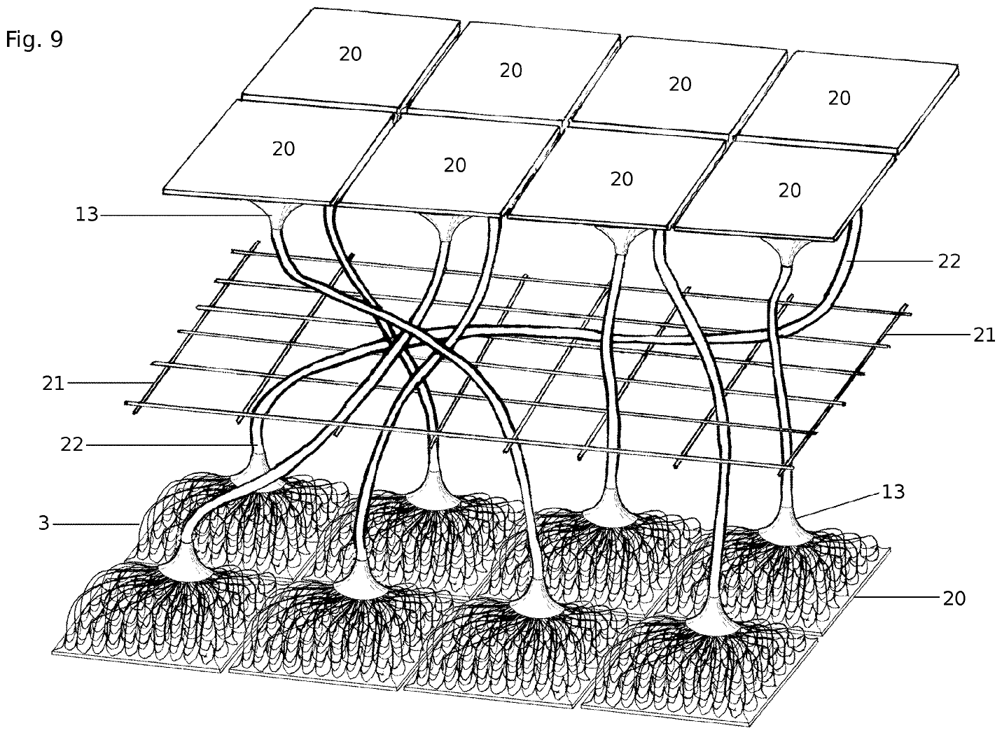

FIG. 9 is an exploded perspective view showing the component of FIG. 8;

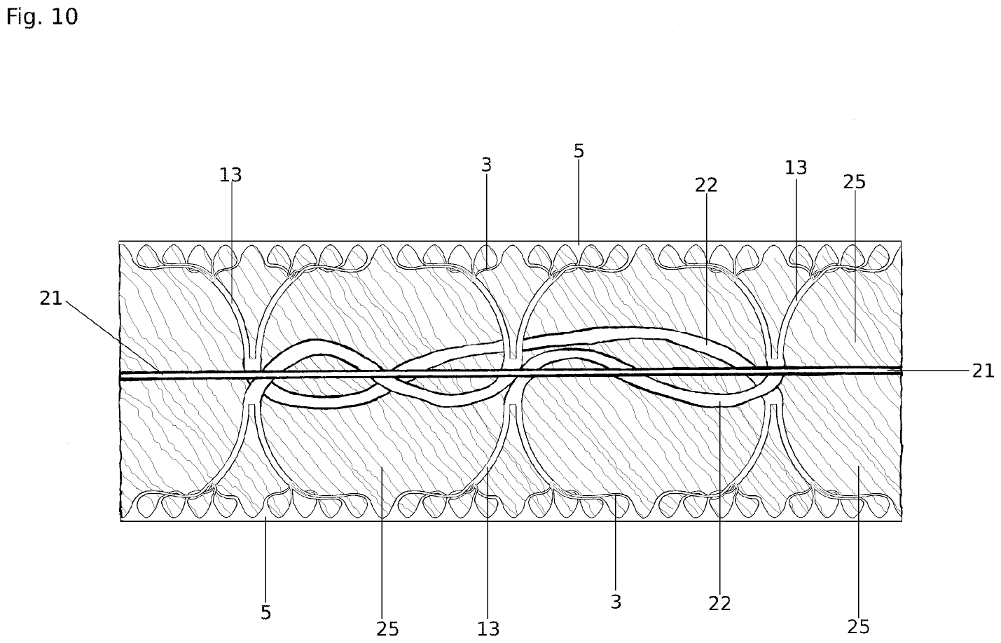

FIG. 10 is a view similar to FIG. 8 of the finished component;

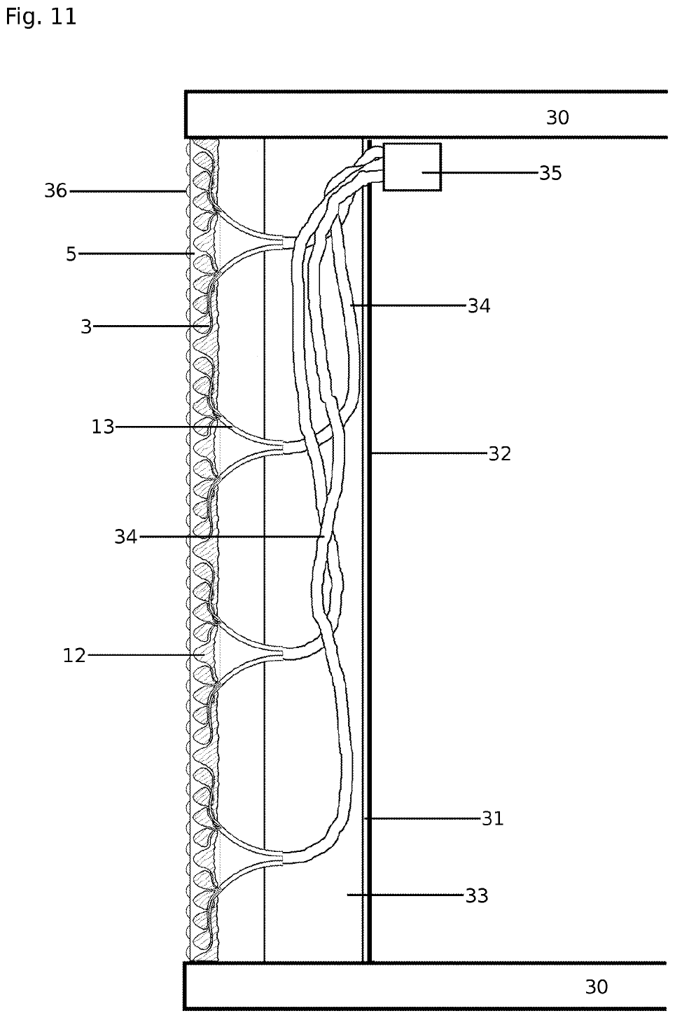

FIG. 11 is a cross-sectional view of a third component in the form of an infill wall panel, prior to the addition of the organic substrate

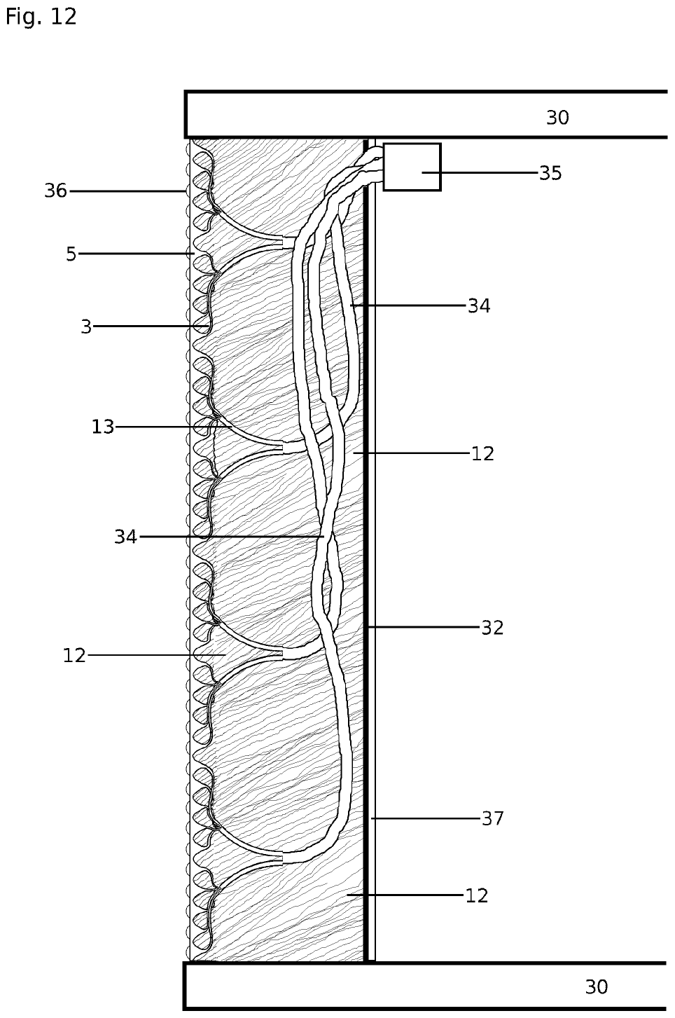

FIG. 12 is a view similar to FIG. 11 showing the finished third component.

FIGS. 1 to 7 illustrate various stages in a method according to a first example of the present invention. As shown in FIG. 1, there is a steel mould 1 in the form of a rectangular tray. The mould can, of course, be of any desired shape. Above the mould 1 is a steel separator 2 into the top of which a bundle of plastic optical fibres 3 are fed. The separator 2 then distributes the fibres 3 in the manner shown in FIG. 1. The fibres 3 are held by a movable clamp 4 as described in greater detail below.

As a first stage in the moulding process, a molten light transmissive numeric plastic such as molten polymethyl methacrylate (PMMA) 5 is provided in the base of the mould 1. This can either be poured in molten form or may be melted in situ.

As shown in FIG. 2, the clamp 4 is then moved downwardly dipping the ends of the optical fibres 3 into the molten plastic. The clamp 4 is then raised as shown in FIG. 3 such that each optical fibre 3 draws some of the plastic 5 up with it from an undulating top to the layer of the plastic which is continuous with the fibres 3.

FIG. 4 shows the introduction of a thin layer of dry organic substrate, added when the molten plastic has almost solidified. As this layer of substrate will be visible through the plastic, flakes of dry leaf litter are particularly suitable, due to their pleasing visual appearance. Once the molten plastic has solidified, the mould is ready to be filled with the organic substrate and the bonding agent.

At this stage, the separator 2 and clamp 4 are raised and the fibres 3 are gathered and cut, forming the component shown in FIG. 5. The ends of the fibres are preferably squeezed together in a hot mould, to form an annulus or other shaped surface 8. Light which hits the outer surface 11 of the light transmitting plastic 5 is channeled through the fibres 3, leaving the component through the surface 8.

This component is then placed in a mould 15, which is then filled with an organic substrate 6 and the bonding agent 10, as shown in FIG. 6. The organic substrate may be leaf litter, wood chips, twigs, weeds, grass cuttings, cardboard, corn husks, rice husks, straw or other agricultural or municipal waste.

The substrate materials should be dried for use. In order to minimize the labour required during preparation, it is not necessary to remove small quantities of soil, mud, dead insects or other low odour contaminates which might naturally be present in the organic substrate. Other inorganic material such as sand, clay, gravel, plastic waste or rubble may be added. This will increase the thermal mass, or the thermal and acoustic insulation provided by the finished product.

Whether or not an inorganic substance has been added, the substrate is preferably poured into a hot tub of mucilage. This is a mixture of water, nutrients, polymers and fire retardant compounds chosen to compliment the substrate. Inorganic components of the substrate may require a more adhesive mucilage and some substrates may require more fire retardant forms of mucilage.

A wire mesh bag can be used to retrieve the substrate from the hot mucilage and the bag can then be placed in a centrifuge to spin off most of the mucilage. Material inoculated with fungal mycelium 10 is then introduced on top of the substrate 6 whereupon the mould is left alone for several weeks to allow time for the substrate to be colonized by the fungal mycelium potentially resulting in full colonization of the component. At one or more point during this process, the substrate and fungal mycelium may be compacted to increase the density of the component, and encourage further mycelium growth. Once it has reached this stage, the contents of the mould are either heated, or chemically treated, to kill the mycelium and prevent further maturation of the bonding agent. The finished component can then be removed from the mould 1, and a trumpet shaped optical junction 13, which concentrates the light leaving the surface 8 into a bundle of optical fibres 14, may be cold welded onto the surface 8. The finished component, complete with optical junction 13, is shown in FIG. 7.

This finished component has a lower surface 11 which is formed of the light transmissive plastic 5 which is linked by a number of the flexible plastic optical fibres 3 which lead back to a second surface 8. The matrix material 12 provides a substrate which has now been substantially colonized by the fungal mycelium, supporting and strengthening the light transmitting plastic 5 and optical fibres 3.

To prevent the drying of mucilage, or the premature maturation of the bonding agent, the mould must either be sealed with a lid, or it must be matured in a high humidity environment.

The structure forms a component which concentrates the light incident on the surface 11 into a more intense light carried by the optical fibres 14. This allows the component to concentrate and direct external light into an internal environment. Alternatively, the component may operate in the opposite sense such that it will produce a more defuse light from a concentrated source. This orientation is suitable, for example, as a light shade.

Many other configurations may be used. For example, the fibres 3 may not be gathered and may extend across the substrate in order to provide a number of smaller “pin points” of light in the upper surface. Alternatively, the fibres 3 may be dispensed with and the plastic layer 5 may, in part, extend fully through the component.

FIGS. 8 to 10 show a second composite component. This is formed of sixteen sub-components 20 each of which is broadly as disclosed in FIGS. 1 to 7. The sub-components 20 are arranged in two arrays of eight which face one another in a back to back arrangement separated by a wire mesh 21. The fibres 3 of a panel on one side of the assembly are connected via a cable 22 to an offset panel 20 such that light can be transmitted through the assembly in a scrambled manner such that no image can be determined. For greater transparency, adjacent panels on the top and bottom layer can be connected together.

As shown in FIG. 8, the sub-assemblies 20 can be placed into a mould 23 with the individual components of fibres 3 and plastic 5 formed as described in relation to the first example. Initially, material inoculated with fungal mycelium is provided at a number of discrete sites 24 in the substrate 6 and is then left to colonize the substrate 6, producing mycelium biocomposite 25 and a finished product shown in FIG. 10. The mycelium for each sub-component 20 will grow into and bond with the mycelium in an adjacent sub-assembly so that the sub-components 20 fuse together.

A third example is shown in FIGS. 11 and 12. This time, the component is formed in situ on a structural frame 30, combining a stud wall frame 31 and the present invention to form a wall of a building. A number of partially complete panels are formed from a light transmitting plastic layer 5, fibres 3 and substrate colonized by mycelium 12, as depicted in FIG. 7. These are erected in an array similar to that shown on the side of FIGS. 8 to 10, spanning between the structural frame 30. Optical junctions 13 are preferably cold welded onto the light emitting surfaces 8 in situ, and optical cables 34 leading from the optical junctions 13 direct the external light to an internal light fitting or diffuser 35. A sheet of flexible plastic 32 is attached to the inner surface defined by the studs 31. This plastic sheet 32 can be opened to allow organic substrate to be poured or sprayed into the cavity 33. Plumbing or electrical cables may also be installed to run through the cavity 33, before it is filled with organic substrate.

The plastic sheet 32 should allow release of gas and water vapour when open, but should maintain high humidity in the cavity 33 when the sheet is closed. Once the required growth has occurred the component is heated in situ, for example using an infra-red LED heater or a hot air blower to briefly raise the temperature of the component, thereby killing the fungus and drying the mucilage. This prevents further maturation of the bonding agent and results in the finished product as shown in FIG. 12. Plasterboard or some other cover 37 is provided to cover the space where the sheet was present. Lenses 36 are formed in the moulding process on the outer face on the component to increase the amount of light captured when the light strikes a surface at an oblique angle.

1. A method of forming a light transitive composite component, the method comprising inserting at least one light transmitting component into a mould;
introducing an organic substrate into the mould;
introducing a bonding agent into the mould,
allowing the bonding agent to mature to bond both the light transmitting component and the organic substrate;
treating the contents of the mould with either heat or chemicals to terminate the maturation process of the bonding agent.

2. A method according to claim 1, wherein the organic substrate comprises one or more of: leaf litter, wood chips, twigs, weeds, grass cuttings, cardboard, agricultural waste, corn husks, rice husks and straw.

3. A method according to claim 1, wherein the bonding agent comprises fungal mycelium.

4. A method according to claim 1, wherein the treatment to terminate the maturation of the bonding agent is the application of heat sufficient to kill the bonding agent.

5. A method according to claim 1, wherein the treatment to terminate the maturation of the bonding agent is the application of fungicidal compounds.

6. A method according to claim 5, wherein the fungicidal; compound is 2-aminoisobutyric acid.

7. A method according to claim 1, wherein the organic substrate is coated with mucilage before being introduced into the mould.

8. A method according to claim 1, further comprising including inorganic material together with the organic substrate.

9. A method according to claim 8, wherein the inorganic material is one or more of sand, clay, gravel, plastic waste or rubble.

10. A method according to claim 1, wherein the at least one light transmitting component is at least one plastic optical fibre.

11. A method according to claim 1, wherein the method comprises forming at least one light transmitting component by moulding a layer of light transmissive plastic at a surface of the mould.

12. A method according to claim 10, wherein the plastic optical fibres are embedded in the layer of light transmissive plastic in the mould.

13. A method according to claim 1, wherein at least one outer surface of the composite component is coating with resin.

14. A method according to claim 1, further comprising at least one step of compressing the fungal mycelium and organic substrate during the stage where the mycelium is growing.

15. A composite component formed of at least one light transmitting component embedded in a matrix of mycelium comprising a mesh of hyphae which are embedded in an organic substrate.

16. A component according to claim 15, wherein the organic substrate comprises one or more of: leaf litter, wood chips, twigs, weeds, grass cuttings, cardboard, agricultural waste, corn husks, rice husks and straw.

17. A component according to claim 15, further comprising a non-organic substrate.

18. A composite according to claim 17, wherein the non-organic substrate is one or more of sand, clay, gravel, plastic waste or rubble.

19. A component according to claim 15, wherein the at least one light transmitting component is at least one plastic optical fibre.

20. A component according to claim 15, wherein a portion of one face of the component is formed as a layer of light transmissive plastic to form the light transmitting component.

21. (canceled)

22. (canceled)
